(19) United States
(12) Patent Application Publication
(10) Pub. No.: US 2026/0199021 A1
(43) Pub. Date: Jul. 16, 2026

(54) SYSTEM AND METHOD FOR TRACKING INTRA-BODY STEERABLE ASSEMBLY

(71) Applicant: Arizona Board of Regents on behalf of Arizona State University, Scottsdale, AZ (US)

(72) Inventors: Hamidreza Marvi, Chandler, AZ (US); Mahdi Ilami, Tempe, AZ (US)

(21) Appl. No.: 19/134,622

(22) PCT Filed: Dec. 6, 2023
(86) PCT No.: PCT/US2023/082678

§ 371 Date: May 30, 2025

Related U.S. Application Data
(60) Provisional application No. 63/386,543, filed on Dec. 8, 2022.

(51) Int. Cl. A61B 34/20; A61B 5/08; A61B 34/10; A61B 34/30; A61B 90/00

(52) U.S. Cl. CPC A61B 34/20; A61B 5/0816; A61B 34/10; A61B 34/30; A61B 90/37; A61B 2034/105; A61B 2034/2061; A61B 2034/305; A61B 2034/306; A61B 2090/3762

(57) ABSTRACT

A system and method for determining positional information of a steerable assembly within a body (optionally comprising an animal (e.g., human) body) utilizes a rigid guide tube having a bore through which the steerable assembly and Fiber Bragg Grating (FBG) sensors, the guide tube having a non-linear shape that is conferred to the steerable assembly and is detectable by a FBG detector to define an origin of the steerable assembly. Continued passage of the steerable assembly through the rigid guide tube at a gate position thereof causes the origin to change with time, and the origin is used in conjunction with the FBG sensors to determine a 3D trajectory of the steerable assembly in the body. The 3D trajectory may be further updated responsive to sensing of relative position and relative orientation between the guide tube and the body.

### STATEMENT OF RELATED APPLICATION(S)

This application claims the benefit of U.S. Provisional Patent Application No. 63/386,543 filed on Dec. 8, 2022, the disclosure of which is hereby incorporated by reference herein in its entirety.

### TECHNICAL FIELD

This disclosure relates generally to assemblies that are steerable (e.g., surgical instruments and borescopes) within a body, and relates more specifically to systems and methods for determining positional information of steerable assemblies.

### BACKGROUND

Various types of minimally invasive surgery involve passing steerable assemblies such as catheters, needles, and endoscopes through an incision or orifice into an animal (e.g., human) body, to perform various ablation, embolization, device placement, and other procedures. Categories of minimally invasive surgeries include endoscopy, laparoscopy, arthroscopy, interventional radiology, etc. Minimally invasive surgery typically has less operative trauma, other complications, and adverse effects than a corresponding open-type surgery (involving a larger incision to permit direct viewing and manipulation of tissue by a surgeon).

Minimally invasive surgeries frequently use image guidance to help surgeons in the localization of the surgical tool. The main imaging techniques include magnetic resonance imaging (MRI), computed tomography (CT), ultrasound, and fluoroscopy. Such tracking devices impose limitations on surgical systems. For example, MRI scanners have a confined space that creates limitations for surgical robots. Additionally, MRI scanners generate strong magnetic fields that render it difficult to utilize ferromagnetic and paramagnetic materials in conjunction with MRI imaging. CT scanning has other disadvantages, such as patient exposure to high doses of radiation (X-rays), disruption of brain imaging by nearby bones, and presence of localized artifacts within images. While attractive for near-surface procedures, ultrasonic imaging provides limited resolution as depth increases, ultrasonic waves are susceptible to being blocked by bones, and artifacts may be common in ultrasound images.

One method to determine position of a steerable assembly within tissue of an animal body without requiring imaging is disclosed in International Publication No. WO 2021/108690 A1, which names the same co-Inventors as the present application. Such publication discloses use of a steerable assembly comprising an elongated structure (e.g., an elongated body structure), an implement arranged at a distal end thereof, and at least one fiber bragg grating (FBG) sensor arranged in or on the elongated structure. Light signals may be supplied to FBG sensors by a FBG driver/detector arranged external to the animal body, wherein reflected light signals received by the FBG driver/detector may be used to determine one or more of force, strain, or shape of the FBG sensors associated with the elongated structure, and thereby used to determine orientation of the elongated structure. The disclosed method further comprises determining a length of insertion of the elongated structure into the tissue of the animal body. The method further comprises using the at least one FBG sensor, sensing one or more conditions indicative of at least one of force, shape, or strain experienced by the FBG sensor(s) during insertion of the elongated structure, determining a three-dimensional trajectory of the steerable assembly from (i) the insertion length, and (ii) the sensed one or more conditions, and superimposing the three-dimensional trajectory (into the animal body) of the steerable assembly on a (previously-constructed) three-dimensional model of the tissue of the animal body. The resulting positional determination does not require real-time imaging of the tissue during insertion of the elongated structure.

There exist limitations of the tracking method disclosed by WO 2021/108690 A1 that may restrict its utility. FBG sensors include fixed sensing points along an optical fiber that can capture the shape of the fiber in three dimensions. Typically, x, y, and z coordinates of each sensing point along the fiber are recorded with respect to a first sensing point. As a first FBG sensing point moves around (i.e., advances), the information of other FBG sensing points will change, thereby causing difficulties in relating the captured shape data to the environment in which the FBG-sensor-containing optical fiber is working. To attempt to address this problem, different approaches may be employed such as fixing the first sensing point, or using additional sensors on an elongated structure that may inhibit maneuverability and/or introduce prohibitive complexity. A further limitation of the tracking method disclosed by WO 2021/108690 A1 is that its accuracy may degrade precipitously due to movement of the animal body.

There also exist applications outside the surgical context for tracking a steerable assembly within a non-animal body—for example, inspection of equipment and structures using borescopes and the like.

In view of the foregoing, the art continues to seek improvement in systems and methods for determining positional information of a steerable assembly within a body (including but not limited to an animal body) to enhance their utility.

### SUMMARY

Aspects of the present disclosure relate to a system and method for determining positional information of a steerable assembly within a body, utilizing a rigid guide tube having a bore through which the steerable assembly having fiber bragg grating (FBG) sensors passes, wherein the rigid guide tube has a non-linear shape that is conferred to the steerable assembly and detectable by a FBG detector to define an origin of the steerable assembly. As the steerable assembly continues to pass through the rigid guide tube at a specific gate position thereof (e.g., optionally a midpoint of the rigid guide tube), the origin of the steerable assembly will change with time, and is used in conjunction with the plurality of FBG sensors to determine three-dimensional (3D) trajectory of the steerable assembly in the body. Optionally, relative position and relative orientation between the rigid guide tube and the body may be sensed, and responsive thereto the determination of 3D trajectory of the steerable assembly may be updated. The steerable assembly comprises an elongated structure and an implement arranged at a distal end thereof, with the plurality of fiber bragg grating sensors arranged in or on the elongated structure. The rigid guide tube is configured to be arranged at a fixed position relative to an opening in the body, wherein the bore thereof is configured to permit passage of the elongated structure. The bore extends between a proximal end and a distal end of the rigid guide tube, wherein at least a portion of a path of the bore between the proximal end and the distal end comprises a non-linear (e.g., curved) path. A FBG detector is configured to receive signals from the plurality of FBG sensors indicative of at least one of force, shape, or strain experienced by the FBG sensors during insertion of the elongated structure into the interior of the body. At least one processor is configured to perform multiple tasks, such as: detecting location of a FBG sensor coincident with a gate position of the rigid guide tube to define an origin of the elongated structure; determining a three-dimensional trajectory of the steerable assembly from (i) the sensed one or more conditions indicative of at least one of force, shape, or strain experienced by the FBG sensors, and (ii) the origin of the elongated structure; and superimposing the three-dimensional trajectory of the steerable assembly on a three-dimensional model of the body. The three-dimensional model may be pre-defined, such as by CT scanning or other means.

In one aspect, the disclosure relates to a method for determining positional information of a steerable assembly within a body, the steerable assembly comprising an elongated structure and an implement arranged at a distal end of the elongated structure, wherein the method comprises multiple steps. The method comprises providing a plurality of fiber bragg grating (FBG) sensors in or on the elongated structure. The method further comprises passing the elongated structure (i) through a rigid guide tube arranged at a fixed position relative to an opening in the body and (ii) into an interior of the body. The method further comprises detecting a location of an FBG sensor coincident with a gate position of the rigid guide tube to define an origin of the elongated structure. The method further comprises sensing one or more conditions indicative of at least one of force, shape, or strain experienced by the FBG sensors during insertion of the elongated structure into the interior of the body. The method further comprises determining a three-dimensional trajectory of the steerable assembly from (i) the sensed one or more conditions indicative of at least one of force, shape, or strain experienced by the FBG sensors, and (li) the origin of the elongated structure. The method further comprises superimposing the three-dimensional trajectory of the steerable assembly on a three-dimensional model of the body.

In certain embodiments, the rigid guide tube comprises a bore extending between a proximal end and a distal end thereof, wherein at least a portion of a path of the bore between the proximal end and the distal end comprises a curved path.

In certain embodiments, the method further comprises sensing relative position and relative orientation between the rigid guide tube and the body, and responsive to such sensing, updating the determination of the three-dimensional trajectory of the steerable assembly.

In certain embodiments, the body comprises an animal body, and the method further comprises: affixing a plurality of motion capture tags to the animal body; sensing position of the plurality of motion capture tags; and adjusting one or more properties of the three-dimensional model of the animal body using the sensed position of the plurality of motion capture tags, and optionally adjusting relative position and/or orientation of the rigid guide tube relative to the animal body.

In certain embodiments, the method further comprises sensing a condition Indicative of respiration rate and/or respiration amplitude of the animal body, and responsive to the sensing, adjusting one or more properties of the three-dimensional model of the tissue of the animal body.

In certain embodiments, the method further comprises providing a visual output of the three-dimensional trajectory of the steerable assembly superimposed on the three-dimensional model of the body.

In certain embodiments, the body comprises an animal body, further comprising performing a computerized tomography (CT) scan of the tissue of the animal body to generate the three-dimensional model.

In certain embodiments, the method further comprises performing one or more tissue imaging steps after or during the passing the elongated body structure into the tissue of the animal body, and responsively updating the three-dimensional model of the tissue of the animal body.

In certain embodiments, the method further comprises affixing the rigid guide tube to the animal body proximate to the opening in the animal body.

In certain embodiments, the opening into an animal body comprises an incision.

In certain embodiments, the steerable assembly comprises a premagnetized material proximate to the distal end; and the method further comprises altering strength and/or position of at least one magnetic field source external to the body to interact with the premagnetized material to effectuate movement of the implement within the body.

In another aspect, the disclosure relates to a system for determining positional information of a steerable assembly within an interior of a body. The system comprises an elongated structure comprising an implement arranged at a distal end thereof, and comprising a plurality of fiber bragg grating (FBG) sensors in or on the elongated structure. The system further comprises a rigid guide tube configured to be arranged at a fixed position relative to an opening in the body, the rigid guide tube comprising comprises a bore extending between a proximal end and a distal end thereof, wherein at least a portion of a path of the bore between the proximal end and the distal end comprises a non-linear path, and the bore is configured to permit passage of the elongated structure. The system further comprises a FBG detector configured to receive signals from the plurality of FBG sensors indicative of at least one of force, shape, or strain experienced by the FBG sensors during insertion of the elongated structure into the interior of the body. The system additionally comprises at least one processor configured to: detect a location of a FBG sensor coincident with a gate position of the rigid guide tube to define an origin of the elongated structure; determine a three-dimensional trajectory of the steerable assembly from (i) the sensed one or more conditions indicative of at least one of force, shape, or strain experienced by the FBG sensors, and (ii) the origin of the elongated structure; and superimpose the three-dimensional trajectory of the steerable assembly on a three-dimensional model of the body.

In certain embodiments, at least a portion of a path of the bore between the proximal end and the distal end comprises a curved path.

In certain embodiments, the body comprises an animal body, and the system further comprises: a plurality of motion capture tags configured to be affixed to the animal body; and a motion capture detector configured to sense position of the plurality of motion capture tags; wherein the at least one processor is further configured to adjust one or more properties of the three-dimensional model of the animal body using the sensed position of the plurality of motion capture tags, and optionally adjust relative position and/or orientation of the rigid guide tube relative to the animal body.

In certain embodiments, the body comprises an animal body, and the system further comprises at least one respiration sensor configured to sense a condition indicative of respiration rate and/or respiration amplitude of the animal body, wherein the at least one processor is further configured to, responsive to such sensing, adjust one or more properties of the three-dimensional model of the tissue of the animal body.

In certain embodiments, the system further comprises a display device configured to provide a visual output of the three-dimensional trajectory of the steerable assembly superimposed on the three-dimensional model of the body.

In certain embodiments, the elongated body structure comprises one or more of a hollow tube, a catheter, an electrical conductor, and a camera.

In another aspect, any two or more features of aspects and/or embodiments disclosed herein may be combined for additional advantage.

### BRIEF DESCRIPTION OF THE DRAWINGS

FIG. 1 schematically illustrates an optical fiber having multiple FBG sensors along its length, wherein each FBG sensor serves as an individual sensing point.

FIG. 2 schematically illustrates a guide tube having a S-shape, through which a flexible device having an optical fiber with multiple FBG sensors passes.

FIG. 3 schematically illustrates an S-shaped guide tube and identifies a predetermined portion of the guide tube that defines a gate position.

FIG. 4 schematically illustrates a flexible device extending through an S-shaped guide tube, with identification of an initial origin (outside the guide tube) defined by a FPG coordinate system and a subsequent origin determined by translation of a FPG sensor to a gate position of the guide tube.

FIG. 5 is a magnified view of the guide tube of FIG. 4, with orthogonal X, Y axes of a coordinate system superimposed on the guide tube thereon at the gate position.

FIG. 6 shows the items of FIG. 4, with superimposed orthogonal X, Y axes of a FPG coordinate system at an initial origin, and with superimposed orthogonal X, Y axes of a rotated coordinate system at the gate position to align with a guide tube coordinate system.

FIG. 7 schematically illustrates interconnections between components of a system for determining positional information of a steerable assembly having an elongated structure (e.g., surgical instrument) and a plurality of FBG sensors, within an interior of a body according to one embodiment, the system further including robotic magnetic manipulators arranged external to the body.

FIG. 8 is a perspective view of a robotic arm incorporating magnets to serve as an end effector to effectuate movement of a steerable assembly with a body (e.g., to manipulate a magnetic needle within tissue of an animal body) according to certain embodiments.

FIG. 9 schematically illustrates a portion of an FBG sensor that may be utilized with a system for determining position of a steerable assembly (e.g., a surgical instrument or borescope) within a body according to certain embodiments.

FIG. 10 is a schematic diagram of a generalized representation of a computer system that can be included as one or more components of a system or method for manipulating tissue during a surgical procedure as disclosed herein.

### DETAILED DESCRIPTION

Aspects of the present disclosure relate to a system and method for determining positional information of a steerable assembly within a body, utilizing a rigid guide tube having a bore through which the steerable assembly having fiber bragg grating (FBG) sensors passes, wherein the rigid guide tube has a non-linear shape that is conferred to the steerable assembly and detectable by a FBG detector to define an origin of the steerable assembly. As the steerable assembly continues to pass through the rigid guide tube at a specific gate position thereof (e.g., optionally a midpoint of the rigid guide tube), the origin of the steerable assembly will change with time, and is used in conjunction with the plurality of FBG sensors to determine three-dimensional (3D) trajectory of the steerable assembly in the body. Optionally, relative position and relative orientation between the rigid guide tube and the body may be sensed, and responsive thereto the determination of 3D trajectory of the steerable assembly may be updated. The steerable assembly comprises an elongated structure and an implement arranged at a distal end thereof, with the plurality of FBG sensors arranged in or on the elongated structure. The rigid guide tube is configured to be arranged at a fixed position relative to an opening in the body, wherein the bore thereof is configured to permit passage of the elongated structure. The bore extends between a proximal end and a distal end of the rigid guide tube, wherein at least a portion of a path of the bore between the proximal end and the distal end comprises a non-linear (e.g., curved) path. A FBG detector is configured to receive signals from the plurality of FBG sensors indicative of at least one of force, shape, or strain experienced by the FBG sensors during insertion of the elongated structure into the interior of the body.

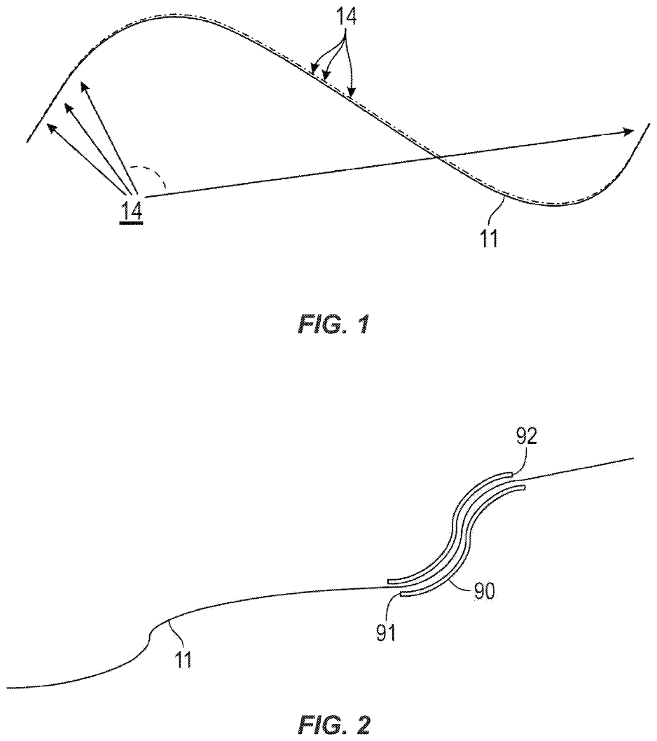

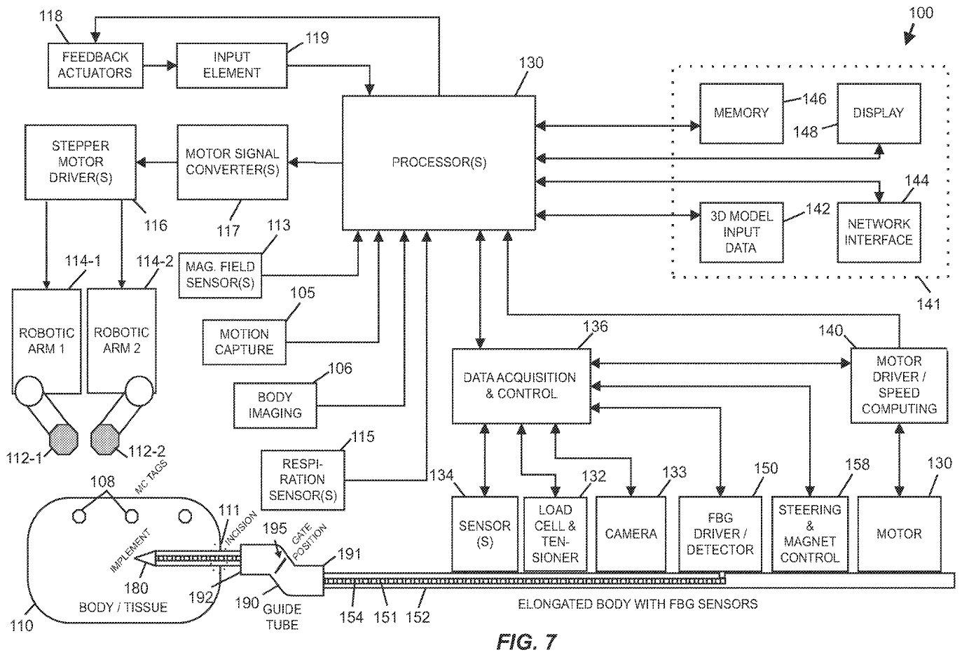

The disclosure relies upon use of FBG sensors, in which an optical fiber has fixed sensing points along its length that permit determination of the shape of the fiber in three dimensions. FIG. 1 schematically illustrates an optical fiber 11 having multiple FBG sensors 14 along its length, wherein each FBG sensor 14 serves as an Individual sensing point. In use, the optical fiber 11 including FBG sensors 14 is Integrated or otherwise coupled with an elongated structure (e.g., of a surgical instrument, a borescope, or the like, such as body 152 shown in FIG. 7) that is subject to being advanced into a body (either an animal body or non-animal body). A FBG detector (coupled with the optical fiber having FBG sensors, such as FBG detector 150 shown in FIG. 7) can record x, y, and z coordinates of each sensing point 14 along the optical fiber 11 with respect to an initial or first sensing point along the optical fiber.

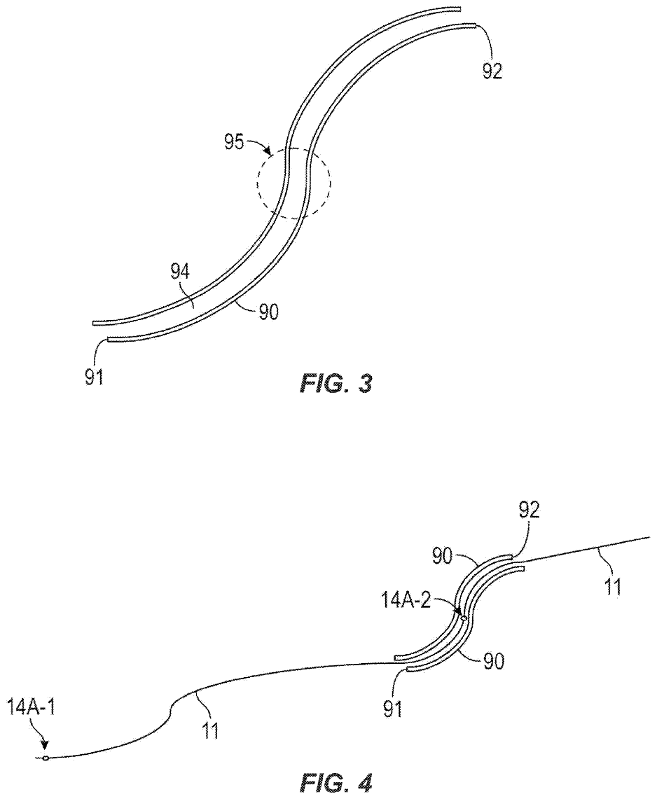

To avoid problems associated with the movement of the first sensing point, the coordinate system of a first sensing point is translated to a tracked (either fixed or moving) location along the optical fiber, wherein this tracked location is defined by a rigid guide tube having a bore through which the optical fiber passes. FIG. 2 shows a guide tube 90 arranged in an S-shape, having a proximal end 91 and a distal end 92 through which a flexible device passes, the flexible device having an optical fiber 11 with multiple FBG sensors (i.e., 14 in FIG. 1, or 154 in FIG. 7). At least a portion of a path defined by a bore (e.g., 94 in FIG. 3) of the rigid guide tube 90 should be non-linear (e.g., curved, such as in a S-shape) to facilitate detection of the guide tube 90. The guide tube 90 is placed at a position that is fixed relative to an opening into a body through which the flexible device (including optical fiber 11) will be inserted. As shown in FIG. 3, a predefined position of the guide tube 90 (with such position optionally being a midpoint between the proximal end 91 and the distal end 92 thereof) defines a gate position 95, wherein presence of a FBG sensor in the bore 94 of the guide tube 90 at the gate position 95 can be detected. FIG. 4 schematically illustrates the optical fiber 11 (having FBG sensors as described previously herein) as part of a flexible device passing from the proximal end 91 through the distal end 92 of an S-shaped guide tube 90. Such figure identifies an initial origin 14A-1 (outside the guide tube 90) defined by a FPG coordinate system corresponding position of an individual FPG sensor (e.g., a leading FPG sensor 14 of FIG. 1). As shown in FIG. 4, when an FBG sensor 14A-2 of the optical fiber 11 is translated to a position with the guide tube 90 coincident with the gate position (e.g., gate position 95 shown in FIG. 3)), the position of the FBG sensor 14-2 become the (new) origin and sets a coordinate system for the optical fiber 11. As an optical fiber is advanced (moved) through the guide tube and a next FBG sensor is detected to be coincident with the gate position of the guide tube, the origin will change to coincide with the next FBG sensor. Restated, different sensing points (i.e., FPG sensors 14 in FIG. 1) will coincide with the gate position (e.g., 95 in FIG. 3) as the optical fiber 11 moves through the guide tube 90, each such sensing point will be considered the new origin at the instant it is so located, and sensed information for the plurality of FBG sensors (14 in FIG. 1) is translated with respect to the instantaneous origin. This method permits free movement of an initial sensing point (14-A-1 in FIG. 4) past the origin without detrimentally affecting subsequent FBG measurements.

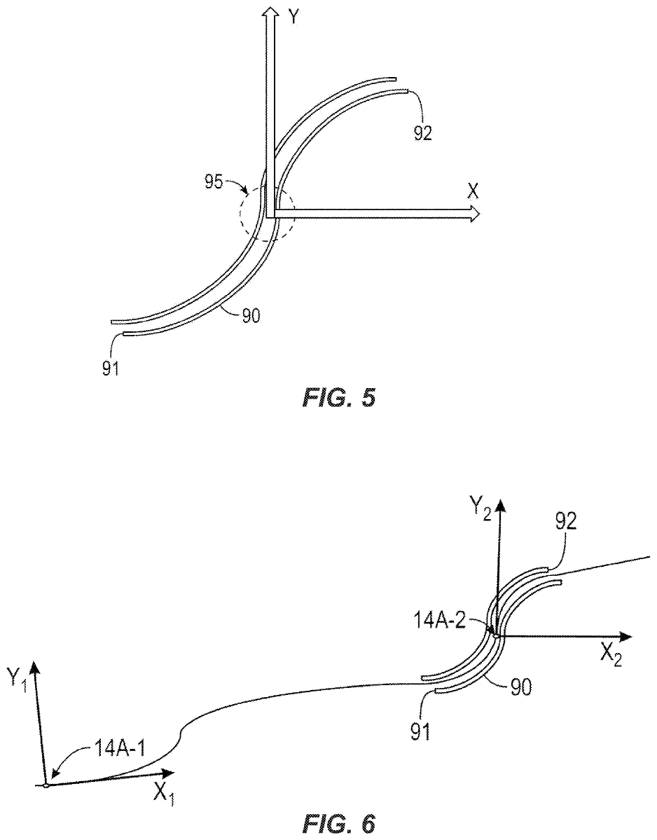

The guide tube 90 defines a new coordinate system. FIG. 5 shows how a guide tube 90 (having an S-shaped configuration according to certain embodiments, spanning from a proximal end 91 to a distal end) defines an XY plane. FIG. 6 shows the items of FIG. 4, with superimposed orthogonal X, Y axes of a FPG coordinate system at an initial origin, and with superimposed orthogonal X, Y axes of a rotated coordinate system at the gate position to align with a guide tube coordinate system. As shown in FIG. 6, a section of optical fiber 11 inside the guide tube 90 may be in a 3D plane depending on the first sensing point. A processor (e.g., 130 in FIG. 7) coupled with an FBG detector (e.g., 150 in FIG. 7) determines the angles between the 3D plane of that section of optical fiber 11 and the guide tube 90. By using a 3D rotation matrix, the processor aligns these two planes, which would not be affected by a first or initial sensing point since this adjustment takes place in real-time.

Although guide tubes having S-shaped configurations are shown in various figures, it is to be recognized that a bore of a guide tube (e.g., bore 94 of guide tube 90 in FIG. 3) may define a path having any suitable shape and configuration, including but not limited to two-dimensional and three-dimensional curved shapes. Preferably, however, at least a portion of a guide tube should be arranged in a non-straight configuration, with one or more curves or turns.

After defining the new origin and coordinate system, it is necessary to determine a relationship between a guide tube and a body (e.g., animal body or non-animal body) into which an elongated structure incorporating FBG sensors is inserted. In certain embodiments, a guide tube can be used in combination with an orientation sensor such as a motion capture camera to determine orientation between the guide tube and the body to be analyzed. In such an instance, both the guide tube and portions of the body to be analyzed may be tagged with optical marking elements (e.g., motion sensor tags). Using signals received from a motion capture camera, a processor can update visualization of an elongated structure bearing FBG sensors, if and when the guide tube or the body to be analyzed should move.

Visualization of a uses a predefined (e.g., pre-captured) 3D image information of the body to be analyzed (e.g., captured by CT scan for an animal (e.g., human) body). Information from a shape-sensing optical fiber having FBG sensors is overlaid on the predefined 3D image information to demonstrate the position and shape of the elongated structure (optionally embodied in a surgical device or borescope) within the body. A processor plots the shape-sensing information of FBG sensing points beyond the gate position and neglects FBG sensing points that have not yet passed through the guide tube. Movements of an animal body (e.g., due to heartbeat and breathing) can also be captured via motion capture, and used by a processor to update the visualization images.

By knowing the length of a guide tube and the number of FBG sensors per unit length of an elongated structure, the number of FBG sensing points inside the bore of the guide tube may be determined. A processor may use the data collected from FBG sensors arranged within the bore of a guide tube to identify the new origin and the rotation matrix. The guide tube may be connected to (or otherwise fixed proximate to) an entrance point (e.g., opening or incision) of a body to be analyzed. If an opening is defined in an animal body, in certain embodiments, the opening may comprise an incision, whereas in certain embodiments the opening may comprise a natural opening such as a mouth or anus. The elongated structure containing the optical fiber with FBG sensors will be inserted into the guide tube before entering body to be analyzed, permitting a processor to define the origin and the coordinate system according to the part of the optical fiber with FBG sensors inside the bore of the guide tube. As the optical fiber with FBG sensors moves inside the bore of the guide tube, a search within the vicinity of the previous origin may be performed to identify the new FBG sensing point that is at the gate position of the guide tube to serve as the new origin.

To measure the traveled distance of an optical fiber having FBG sensors, a record may be maintained of the previous origin and the current origin. By knowing the distance between FBG sensing points and the number of FBG sensing points between the previous origin and the current origin, the traveled distance can be calculated.

Embodiments of the present disclosure permit determination of positional information of a steerable assembly arranged within a body to be analyzed, wherein the body may comprise an animal body or a non-animal body, and the steerable assembly may be moved by either pushing from a base portion or magnetically pulled via a tip portion thereof using a magnetic force generator external to the body to be analyzed. The steerable assembly may include an optical fiber and a plurality of FBG sensors as described herein.

In certain embodiments, an elongated body such as a surgical instrument may be steered via pushing, by exploiting asymmetric forces on an instrument (e.g., needle) tip during insertion. As an instrument tip is pushed forward through tissue, it also moves slightly sideways, motivated by the radial component of the force acting on the tip. The magnitude of this sideways movement depends on the tip geometry, tip stiffness, tissue stiffness, bevel angle, and other properties of the instrument tip-tissue interactions. The instrument (or an associated tubular structure connected to the needle) is rotated at the base to control the orientation of the tip, thus rotating the direction of the asymmetric force and permitting the trajectory of the instrument tip to be controlled.

In certain embodiments, an elongated body may constitute a surgical instrument having a magnetically responsive tip and may be steered via magnetic pulling, by being used in conjunction with an instrument needle steering apparatus and method that alters strength and/or position of at least one magnetic field source (e.g., generated by one or more end effectors such as one or more robotic arm(s)) external to an animal body to interact with the instrument tip inserted into the animal body to effectuate movement of the instrument within the animal body. A conventional elongated structure (e.g., shaft) of the surgical instrument may be replaced by an elastic shaft that is not load-bearing. By pulling the instrument tip through tissue using externally applied magnetic forces instead of pushing at the base of a load-bearing shaft supporting a needle, any concern of shaft buckling is eliminated by avoiding formation of compression stresses in the shaft. Additional details regarding magnetic pulling of a surgical instrument through tissue are disclosed in International Publication No. WO 2021/108690 A1, which is hereby incorporated by reference herein.

FIG. 7 schematically illustrates components of a system 100 for determining positional information of a steerable assembly having an elongated structure 152 within an interior of a body 110 (optionally comprising an animal body, including but not limited to a human body) according to one embodiment. At lower left, an elongated structure (e.g., surgical instrument) 152 extends through an opening or incision 111, with a portion thereof positioned within a body 100 to be analyzed. The elongated structure (e.g., surgical instrument) 152 terminates at a tip 180 within the body 110, with the tip 180 optionally comprising one or more of a tool or other implement, a camera, and a premagnetized element, wherein any (or all) of the foregoing elements may be selectively deployed in certain embodiments. The elongated structure 152 further comprises a plurality of fiber bragg grating (FBG) sensors 154 associated with an optical fiber 151 arranged in or on the elongated structure 152. Robotic manipulators 114-1, 114-2 each having an associated magnetic field source 112-1, 112-1 are positioned external to the body 110. The robotic manipulators 114-1, 114-2 may comprise robotic actuators (e.g., robotic arms, such as 6-degree-of-freedom (6DOF) robotic arms) arranged external to the body 110 to be analyzed (e.g., animal body) to apply at least one magnetic field to control and effectuate movement of the elongated structure 152 within the body 110. The robotic manipulators 114-1, 114-2 may be controlled by stepper motor drivers 116 and a processor 130 (e.g., integrated with a microcomputer in certain embodiments), wherein one or more intermediately arranged motor signal converters 117 may also be provided. Desired poses of the robotic manipulators 114-1, 114-2 may be calculated by the processor 130 and supplied to the stepper motor drivers 116 to control movement of the robotic manipulators 114-1, 114-2. Movement of one or more magnetic end effectors 112-1, 112-2 (which may be embodied in permanent magnet materials, ferromagnetic materials, or electromagnets) may be used to pull a magnetic portion (e.g., tip 180) of the elongated structure 152 through the body 110 to be analyzed.

With continued reference to FIG. 7, the elongated body 152 with an optical fiber 151 having multiple FBG sensors 154 extends through a guide tube 190 having a gate position 195 (arranged between a proximal end 191 and a distal end 192 of the guide tube 190) and through an opening or incision 111 into the body 110 to be analyzed. A FBG driver/detector 150 is coupled with FBG sensors 154 of the optical fiber 151 to facilitate FBG measurement.

A user input device 119 controllable by user manipulation is arranged to permit control of the magnetic end effectors 112-1, 112-2. One or more feedback actuators 118 may be configured to supply haptic feedback to the user through the user input device 119 (e.g., proportional to one or more of magnetic field strength, magnetic field direction, tissue displacement, tissue density, deviation from desired trajectory, or the like). One example of a user input device 119 is a joystick, which may be provided in single or dual forms, optionally augmented with various items such as triggers, buttons, dials, and the like. A camera and/or optical fiber (coupled to camera imager 133) associated with the elongated body structure 152 may be provided within the body 110 (optionally within a surgical field for an animal body 110, such as proximate to a surgical tool at a top 180 of the elongated body 152) to enable visualization, such as by using one or more displays 148, whether in stand-alone or wearable (e.g., headset) form.

With continued reference to FIG. 7, FBG sensors 154 associated with an optical fiber 111 are provided in or on the elongated body structure 152, and are inserted through the guide tube 190 into the body 110 to be analyzed. Light signals may be supplied to FBG sensors 154 by a FBG driver/detector 150 arranged external to the body 110 to be analyzed. Reflected light signals received by the FBG driver/detector 150 may be used to determine one or more of force, strain, or shape of FBG sensors 154 associated with the elongated body structure 152, and thereby used to determine orientation of the elongated body structure 152.

In certain embodiments, a tracking subsystem for the elongated body 152 may include a DC motor 130 having a rotatable spool coupled thereto, a load cell and tensioner 132, and a rotary encoder (optionally integrated into a motor driver/speed computing element 140 coupled to the DC motor 130). The foregoing items may be mounted on a moveable support structure (not shown), such as a platform mounted on linear guides that enable one-directional (e.g., horizontal) translation in one direction. One example of a moveable support structure that may be used is shown in International Publication No. WO 2021/108690 A1, with the disclosure thereof being hereby incorporated by reference herein. A portion of the elongated body structure 152 may be wrapped on the spool coupled with a shaft of the DC motor 130. The load cell 132 (or alternatively a force sensor) may be used to measure tensile or compressive loads applied to the moveable support structure, wherein the processor 130 may be used in combination with the rotary encoder (e.g., within motor driver/speed computing element 140) to calculate rotational velocity of the motor 130, which may be used to calculate insertion depth of the elongated body 152 in the body 110. Measurements from the load cell 132 may be used to calculate tension applied to the elongated structure 152. A data acquisition device 136 sends control inputs to a motor driver 140 that supplies power to the DC motor 130 to which the elongated structure 152 is coupled.

In certain embodiments, the DC motor 130 may be used to provide controlled releasement of the elongated body structure 152 from a spool of the motor 130. The processor 130 may be used to compare an output signal of at least one sensor 134 configured to sense a condition indicative of at least one of (i) position of a moveable support structure or (ii) pulling force applied to a moveable support structure, and configured to generate at least one output signal. In certain embodiments, operation of the DC motor 130 may be controlled to adjust a feed rate of a length of elongated body structure 152 from a rotatable spool of the motor 130 responsive to comparison of the output signal to the desired range of output signal values. For example, if tension on the elongated body structure 152 is too high, then in certain embodiments, operation of the DC motor 130 may be controlled to increase the releasement rate of the elongated body structure 152 from the rotatable spool of the motor. In certain embodiments, operation of the DC motor 130 may be controlled to reverse rotational direction of the motor 130 responsive to comparison of the output signal to the desired range of output signal values.

In certain embodiments, a three-dimensional (3D) model of the body 110 (e.g., tissue of an animal body) is generated before a steerable assembly including elongated body 152 (e.g., surgical instrument) is supplied to tissue of the animal body. Such a 3D model may be generated by any suitable imaging device, such as a MRI, CT, ultrasound, fluoroscopy, or other imaging device. The 3D model, optionally received via a network interface 144 and/or generated from 3D model input data 142 as part of a 3D model interaction subsystem 141, may be stored to memory 146 accessible to at least one processor 130, in preparation for receiving 3D trajectory information of a steerable assembly (including the elongated body 152) for superimposition onto the 3D model. This 3D trajectory information may be determined by directly by imaging, or inferentially from a detected length of insertion of the elongated structure 152 into the animal body 110, in combination with a recorded directionality of a magnetic field applied (by magnetic effectors 112-1, 112-2) to a premagnetized material (e.g., magnetic tip 180) associated with the elongated body 152, optionally embodied in a surgical instrument.

In certain embodiments, insertion length of the elongated body structure 152 may be determined (or supplemented) by sensing position or velocity of a shaft of the DC motor 130 controlling releasement of the elongated body structure 152 during insertion of the elongated body structure 152 into the body 110. In certain embodiments, position or velocity of a shaft of the motor 130 may be sensed with a rotary encoder, which may be integrated into a motor driver/speed computing element 140. In certain embodiments, insertion length of the elongated body structure 152 may be determined by sensing linear position or displacement of at least a portion of the elongated body structure 152, such as by using a linear encoder (not shown) arranged between a spool coupled to the motor 130 and the body 110 to be analyzed.

In certain embodiments, recording of directionality of a magnetic field applied to the elongated body 152 in the body 110 to be analyzed comprises recording control signals supplied to the stepper motor drivers 116 coupled with the robotic manipulators 114-1, 114-2 configured to adjust position of magnetic end effectors 112-1, 112-1 configured to apply one or more magnetic fields to a tip 180 of the elongated body 152. In certain embodiments, recording of directionality of the magnetic field may comprise, or be supplemented by, collecting signals received from one or more magnetic field sensors 113. In certain embodiments, one or more magnetic field sensors 113 may be positioned proximate to the body 110 into which the elongated body 152 is inserted.

In certain embodiments, a condition indicative of respiration rate and/or respiration amplitude of an animal body 110 may be sensed (e.g., using respiration sensors 115 and/or a ventilator or one or more chest sensors), and responsive to the such sensing, a 3D model of the animal body 110 (storable in memory 146) may be updated, and/or position of the magnetic end effectors 114-1, 114-2 may be adjusted. If the body 110 comprises an animal body arranged in a lying position, the foregoing control scheme may be used to maintain constant distance in the vertical direction between the tissue of the animal body 110 and the magnetic end effectors 114-1, 114-2 so that a constant magnetic force is applied on a premagnetized needle at a tip 180 of the elongated body 152.

In certain embodiments, motion capture tags 108 may be provided on the body 110 to be analyzed and on the guide tube 190, and a motion capture sensor 105 may be used to establish positions of the body 110 and the guide tube 190. If a position or orientation of the body 110 should change, then a 3D model of the body (e.g., storable in memory 146) may be updated accordingly.

While continuous imaging of a body 110 to be analyzed is not required according to methods disclosed herein, in certain embodiments a body imaging apparatus 106 may be provided to periodically permit imaging of the body 110 and inserted portions of the elongated structure 152, as may be useful to confirm and/or correct FBG-calculated positional information derived from the FBG sensors 154 and FBG detector 150.

In certain embodiments, the system 100 may be configured to receiving signals for linear translation of an elongated body (for determining insertion depth of the elongated body structure 152) and signals for movement of the robotic manipulators 114-1, 114-2 (for determining magnetic field direction) and processing the signals for forwarding to a computer processor 130 for superimposition of 3D trajectory of the elongated body 152 (e.g., optionally embodied in a surgical instrument) on a previously generated 3D model of tissue of an animal body 110 into which the elongated body 152 inserted.

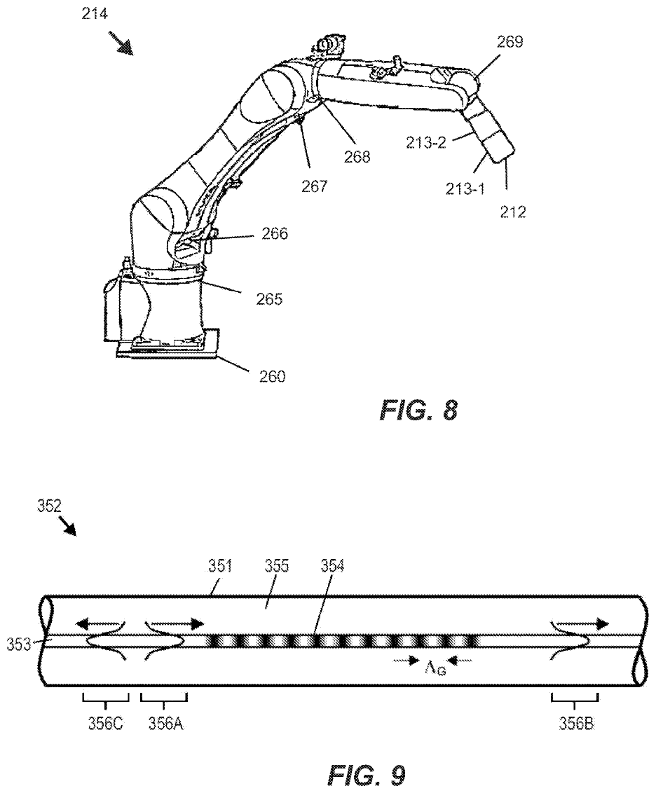

FIG. 8 is a perspective view of a robotic arm 214 incorporating magnets 213-1, 213-2 (e.g., permanent magnets or electromagnets) to serve as an end effector 212 to effectuate movement of a steerable assembly including a magnetic needle within tissue of an animal body according to certain embodiments. In certain embodiments, the magnets 213-1, 213-2 may be, or may be controlled to be, of the same polarity or opposing polarities. The robotic arm 214 is mountable to a support surface 260 and includes multiple joints 265-269 to provide numerous degrees of freedom for movement of the robotic arm 214 relative to tissue of an animal body (e.g., 110 in FIG. 7) in order to effectuate movement of an implement including a premagnetized portion (e.g., needle tip) of a surgical instrument (e.g., magnetic tip 180 in FIG. 7) within tissue of the animal body, and/or to effectuate movement of a tissue anchor (not shown) within the animal body. In certain embodiments, the robotic arm 214 may be used initially to move an implement within tissue of the animal body, and thereafter to manipulate a tissue anchor.

FIG. 9 is a schematic view illustration of a portion of a fiber bragg grating (FBG) sensor 352 that may be utilized with a system for determining position of a steerable assembly (e.g., a surgical instrument or borescope) within a body according to certain embodiments. The FBG sensor 352 is embodied in an optical fiber 351 having a core 353 surrounded by cladding 355. A portion of the core 353 constitutes an index modulation region 354 in which an index of refraction of glass material of the core 353 periodically varies. When an input signal 356A (having a propagating core mode) is transmitted through the core 353 and reaches the index modulation region 354, one spectral portion of the input signal is reflected to produce a reflected signal 356C, while another spectral portion is transmitted through the Index modulation region 354 to provide a transmitted signal 356B. The reflected signal 356C may be detected by a light detector associated with a FBG driver/detector unit (not shown), and analyzed to determine one or more of force, strain, or shape experienced by the FBG sensor 352. In certain embodiments, one or more FBG sensors may be arranged in or on an elongated body structure of a steerable assembly, wherein an index modulation region may be provided proximate to a magnetic needle affixed to the elongated structure.

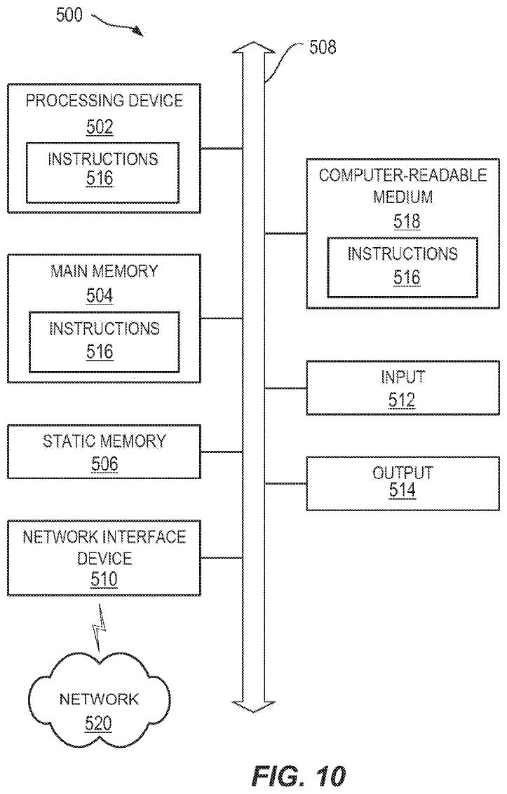

FIG. 10 is schematic diagram of a generalized representation of a computer system 500 that can be included as one or more components of a system or method for determining positional information of a steerable assembly within a body as disclosed herein, according to one embodiment. The computer system 500 may be adapted to execute instructions from a computer-readable medium to perform these and/or any of the functions or processing described herein.

The computer system 500 may include a set of instructions that may be executed to program and configure programmable digital signal processing circuits for supporting scaling of supported communications services. The computer system 500 may be connected (e.g., networked) to other machines in a local area network (LAN), an intranet, an extranet, or the Internet. While only a single device is illustrated, the term “device” shall also be taken to include any collection of devices that individually or jointly execute a set (or multiple sets) of instructions to perform any one or more of the methodologies discussed herein. The computer system 500 may be a circuit or circuits included in an electronic board or card, such as a printed circuit board (PCB), a server, a personal computer, a desktop computer, a laptop computer, a personal digital assistant (PDA), a computing pad, a mobile device, or any other device, and may represent, for example, a server or a user's computer.

The computer system 500 in this embodiment includes a processing device or processor 502, a main memory 504 (e.g., read-only memory (ROM), flash memory, dynamic random access memory (DRAM), such as synchronous DRAM (SDRAM), etc.), and a static memory 506 (e.g., flash memory, static random access memory (SRAM), etc.), which may communicate with each other via a data bus 508. Alternatively, the processing device 502 may be connected to the main memory 504 and/or static memory 506 directly or via some other connectivity means. The processing device 502 may be a controller, and the main memory 504 or static memory 506 may be any type of memory.

The processing device 502 represents one or more general-purpose processing devices, such as a microprocessor, central processing unit (CPU), or the like. In certain embodiments, the processing device 502 may be a complex instruction set computing (CISC) microprocessor, a reduced instruction set computing (RISC) microprocessor, a very long instruction word (VLIW) microprocessor, a processor implementing other instruction sets, or other processors implementing a combination of instruction sets. The processing device 502 is configured to execute processing logic in instructions for performing the operations and steps discussed herein.

The computer system 500 may further include a network interface device 510. The computer system 500 may additionally include at least one input 512, configured to receive input and selections to be communicated to the computer system 500 when executing instructions. The computer system 500 also may include an output 514, including but not limited to a display, a video display unit (e.g., a liquid crystal display (LCD) or a cathode ray tube (CRT)), an alphanumeric input device (e.g., a keyboard), and/or a cursor control device (e.g., a mouse).

The computer system 500 may or may not include a data storage device that includes instructions 516 stored in a computer readable medium 518. The instructions 516 may also reside, completely or at least partially, within the main memory 504 and/or within the processing device 502 during execution thereof by the computer system 500, the main memory 504 and the processing device 502 also constituting computer readable medium. The instructions 516 may further be transmitted or received over a network 520 via the network interface device 510.

While the computer readable medium 518 is shown in an embodiment to be a single medium, the term “computer-readable medium” should be taken to include a single medium or multiple media (e.g., a centralized or distributed database, and/or associated caches and servers) that store the one or more sets of instructions. The term “computer readable medium” shall also be taken to include any medium that is capable of storing, encoding, or carrying a set of instructions for execution by the processing device and that cause the processing device to perform any one or more of the methodologies of the embodiments disclosed herein. The term “computer readable medium” shall accordingly be taken to include, but not be limited to, solid-state memories, an optical medium, and/or a magnetic medium.

Those skilled in the art will recognize improvements and modifications to the preferred embodiments of the present disclosure. All such improvements and modifications are considered within the scope of the concepts disclosed herein and the claims that follow. Any of the various features and elements as disclosed herein may be combined with one or more other disclosed features and elements unless indicated to the contrary herein.

Those skilled in the art will recognize improvements and modifications to the preferred embodiments of the present disclosure. All such improvements and modifications are considered within the scope of the concepts disclosed herein and the claims that follow. Any of the various features and elements as disclosed herein may be combined with one or more other disclosed features and elements unless indicated to the contrary herein.

1. A method for determining positional information of a steerable assembly within a body, the steerable assembly comprising an elongated structure and an implement arranged at a distal end of the elongated structure, the method comprising:
providing a plurality of fiber bragg grating sensors in or on the elongated structure;
passing the elongated structure (i) through a rigid guide tube arranged at a fixed position relative to an opening in the body and (ii) into an interior of the body;
detecting a location of a fiber bragg grating sensor coincident with a gate position of the rigid guide tube to define an origin of the elongated structure;
using the plurality of fiber bragg grating sensors, sensing one or more conditions indicative of at least one of force, shape, or strain experienced by the fiber bragg grating sensors during insertion of the elongated structure into the interior of the body;
determining a three-dimensional trajectory of the steerable assembly from (i) the sensed one or more conditions indicative of at least one of force, shape, or strain experienced by the fiber bragg grating sensors, and (ii) the origin of the elongated structure; and
superimposing the three-dimensional trajectory of the steerable assembly on a three-dimensional model of the body.

2. The method of claim 1, wherein the rigid guide tube comprises a bore extending between a proximal end and a distal end thereof, wherein at least a portion of a path of the bore between the proximal end and the distal end comprises a curved path.

3. The method of claim 1, further comprising sensing relative position and relative orientation between the rigid guide tube and the body, and responsive to such sensing, updating the determination of the three-dimensional trajectory of the steerable assembly.

4. The method of claim 1, wherein the body comprises an animal body, and the method further comprises:
affixing a plurality of motion capture tags to the animal body;
sensing position of the plurality of motion capture tags; and
adjusting one or more properties of the three-dimensional model of the animal body using the sensed position of the plurality of motion capture tags, and optionally adjusting relative position and/or orientation of the rigid guide tube relative to the animal body.

5. The method of claim 4, further comprising sensing a condition indicative of respiration rate and/or respiration amplitude of the animal body, and responsive to the sensing, adjusting one or more properties of the three-dimensional model of the tissue of the animal body.

6. The method of claim 1, wherein the method further comprises providing a visual output of the three-dimensional trajectory of the steerable assembly superimposed on the three-dimensional model of the body.

7. The method of claim 1, wherein the body comprises an animal body, further comprising performing a computerized tomography (CT) scan of the tissue of the animal body to generate the three-dimensional model.

8. The method of claim 1, wherein the body comprises an animal body, further comprising performing one or more tissue imaging steps after or during the passing the elongated body structure into the tissue of the animal body, and responsively updating the three-dimensional model of the tissue of the animal body.

8. The method of claim 1, wherein the body comprises an animal body, and the method further comprises affixing the rigid guide tube to the animal body proximate to the opening in the animal body.

9. The method of claim 8, wherein the opening in the animal body comprises an incision.

10. The method of claim 1, wherein:
the steerable assembly comprises a premagnetized material proximate to the distal end; and
the method further comprises altering strength and/or position of at least one magnetic field source external to the body to interact with the premagnetized material to effectuate movement of the implement within the body.

11. A system for determining positional information of a steerable assembly within an interior of a body, the system comprising:
an elongated structure comprising an implement arranged at a distal end thereof, and comprising a plurality of fiber bragg grating sensors in or on the elongated structure;
a rigid guide tube configured to be arranged at a fixed position relative to an opening in the body, the rigid guide tube comprising comprises a bore extending between a proximal end and a distal end thereof, wherein at least a portion of a path of the bore between the proximal end and the distal end comprises a non-linear path, and the bore is configured to permit passage of the elongated structure;
a fiber bragg grating detector configured to receive signals from the plurality of fiber bragg grating sensors indicative of at least one of force, shape, or strain experienced by the fiber bragg grating sensors during insertion of the elongated structure into the interior of the body; and
at least one processor configured to:
detect a location of a fiber bragg grating sensor coincident with a gate position of the rigid guide tube to define an origin of the elongated structure;
determine a three-dimensional trajectory of the steerable assembly from (i) the sensed one or more conditions indicative of at least one of force, shape, or strain experienced by the fiber bragg grating sensors, and (ii) the origin of the elongated structure; and
superimpose the three-dimensional trajectory of the steerable assembly on a three-dimensional model of the body.

12. The system of claim 11, wherein at least a portion of a path of the bore between the proximal end and the distal end comprises a curved path

13. The system of claim 11, wherein the body comprises an animal body, and the system further comprises:
a plurality of motion capture tags configured to be affixed to the animal body; and
a motion capture detector configured to sense position of the plurality of motion capture tags;
wherein the at least one processor is further configured to adjust one or more properties of the three-dimensional model of the animal body using the sensed position of the plurality of motion capture tags, and optionally adjust relative position and/or orientation of the rigid guide tube relative to the animal body.

14. The system of claim 11, wherein the body comprises an animal body, and the system further comprises at least one respiration sensor configured to sense a condition indicative of respiration rate and/or respiration amplitude of the animal body, wherein the at least one processor is further configured to, responsive to such sensing, adjust one or more properties of the three-dimensional model of the tissue of the animal body.

15. The system of claim 11, further comprising a display device configured to provide a visual output of the three-dimensional trajectory of the steerable assembly superimposed on the three-dimensional model of the body.

16. The system of claim 11, wherein the elongated body structure comprises one or more of a hollow tube, a catheter, an electrical conductor, and a camera.
